(19) United States
(12) Patent Application Publication
(10) Pub. No.: US 2006/0147954 A1
(43) Pub. Date: Jul. 6, 2006

(54) Novel probe and its use in bioaffinity assays

(75) Inventors: Ville Laitala, Turku (FI); Alice Ylikoski, Piikkio (FI)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: WALLAC OY, Turku (FI)

(21) Appl. No.: 11/252,851

(22) Filed: Oct. 19, 2005

Related U.S. Application Data
(60) Provisional application No. 60/619,700, filed on Oct. 19, 2004.

(51) Int. Cl. C12Q 1/68

(52) U.S. Cl. 435/6

(57) ABSTRACT

The invention concerns a labeled probe for use in bioaffinity assays based on time-resolved fluorescence resonance energy transfer, wherein said probe comprises an energy donor as well as an energy acceptor, and the energy transfer signal of the acceptor is to be measured. The invention concerns also a homogeneous time-resolved fluorescence resonance energy transfer bioaffinity assay based on the use of said probe.

### FIELD OF THE INVENTION

This present invention relates to a labeled probe for use in bioaffinity assays based on time-resolved fluorescence resonance energy transfer, wherein said probe comprises an energy donor as well as an energy acceptor, and the energy transfer signal of the acceptor is aimed to be measured. The invention concerns also a homogeneous time-resolved fluorescence resonance energy transfer bioaffinity assay based on the use of said probe. The analyte specific recognition reaction between the probe and the analyte causes a change in the energy transfer signal of the probe. The energy transfer signals of the reacted and non-reacted probes are distinguished based on the lifetime of the signals.

### BACKGROUND OF THE INVENTION

The publications and other materials used herein to illuminate the background of the invention, and in particular, cases to provide additional details respecting the practice, are incorporated by reference.

Förster type of non-radiative dipole-dipole energy transfer (Förster (1948) Ann. Physik., 6, 55) takes place between two molecules in condition where their energies (emission of donor (D) with absorption of acceptor (A)) overlap and they are located at a suitable distance from each other. The energy transfer requires a proper orientation of the oscillations of the molecules. The energy transfer efficiency E is given in the equation E=1/(1+r⁶/R₀⁶). in which r means the distance between the donor and the acceptor, and R₀ is a distance parameter characteristic of the donor-acceptor pair and the medium between them. The usable distance scale for FRET experiments using conventional donor-acceptor pairs is ˜10-100 Å.

Fluorescence resonance energy transfer (FRET), also called luminescence resonance energy transfer (LRET) when lanthanide donors are used, has found extensive applications both in basic research and bioanalytical technologies as a platform for homogenous assays and as a spectroscopic ruler to measure distances in biomolecules. Ullman was the first to describe application of Förster type non-radiative energy transfer in homogenous bioanalytical assays based on antibody recognition reaction (Ullman, Scharzberg and Rubenstein (1967) J. Biol. Chem., 251: 4172) and a great number of suitable donor-acceptor probe pairs have since been developed and applied in immunoassays (for a review see I Hemmila, Applications of Fluorescence in Immunoassays, Wiley, NY, 1991, chapter 8.3.4).

Time-resolved (TR) fluorometry (time resolution in time-domain at micro- or millisecond range) is an excellent measuring regime for homogenous assays because it can totally discriminate against ns-lifetime background fluorescence caused by organic compounds and light scattering. Suitable donors for TR-FRET measurements include among others lanthanide chelates (cryptates) and some other metal ligand complexes, which can have fluorescent lifetime in the micro-millisecond time region, and which therefore also allow the energy transfer to occur in micro-millisecond time region. This enables the time-resolved detection of the FRET-signal. Especially lanthanides and their fluorescent complexes have established a strong position as donors in TR-FRET measurements. Fluorescent lanthanide chelates have been used as energy donors already since 1978 by Stryer, Thomas and Meares (For example see Thomas et al. (1978) Proc. Natl. Acad. Sci., 75: 5746) and since then a number of homogenous TR-FRET based assays have been described and patented (Mathis (1995) Clin. Chem., 41, 1391; Selvin et al. (1994) Proc. Natl. Acad. Sci., USA, 91, 10024) with their limitations and drawbacks.

When certain molecules of interest are screened in a large scale, e.g. in drug screening, homogeneous assays are usually preferred because they are easier to carry out and automate than traditional multi-phased heterogeneous assays. A very common way to carry out a FRET (or TR-FRET) based homogeneous assay is to use separate donor and acceptor labeled binding partners (e.g. labeled antibodies), which bind to the analyte molecule and form an analyte specific energy transfer complex. Another very common way to carry out a FRET (or TR-FRET) based homogeneous assay is to use the competitive assay format, which utilizes for example labeled antibody labeled antigen pair and the addition of unlabeled antigen (analyte) causes a change in the energy transfer signal. Also other embodiments exist but very often two separate labeled reagents are essential to carry out the assay. This increases the number of necessary assay steps and in some cases can also have influence on the sensitivity of the assay, if suitable binding partners with high affinity are not available.

Some energy transfer based methods, in which only one labeled component is needed for obtaining the analyte specific response has been published and patented. These methods are focused on the detection of nucleic acid sequences and utilize dual labeled (donor and acceptor) target-specific probes. These methods use an interacting FRET label pair usually consisting of a fluorescent reporter dye and non-fluorescent acceptor label. A fluorescent acceptor label is also possible.

The TaqMan method is described in Gelfand et al. U.S. Pat. No. 5,210,015 and Livak et al. U.S. Pat. No. 5,538,848. The TaqMan method utilizes dual labeled oligonucleotide probes, in which both the fluorescent reporter dye and the quencher are attached in the same oligonucleotide and the donor signal change in the assay is based on Taq DNA polymerase cleavage of the hybridized probe. This cleavage provides a way to separate the quencher label and the fluorescent reporter dye.

Another method utilizing a dual labeled oligonucleotide probe is the so-called molecular beacon, which is described in U.S. Pat. No. 5,925,517. A molecular beacon is an oligonucleotide whose end regions hybridize with one another in the absence of target but are separated if the central region of the probe hybridizes to its complementary target sequence. The intramolecular hybrid formed by the end regions brings the interacting labels very close to each other and the formation of the rigid probe-target hybrid provides a way to separate the interacting labels, which is the basis of the signal change in the assay.

Both the TaqMan and Molecular beacon methods are quench-based assays, in which the changes in the quenching of the fluorescent reporter dye (donor) is monitored during the assay. In Molecular beacon system, certain additional hybridization procedures (self hybridization) are required to separate the signals from free (not bound to the target) and bound probes. The TaqMan method requires enzymatic activity, which allows the signal to rise. These probes are also limited only to be used in nucleic acid detection.

Moreover, a TR-FRET method utilizing a large protein complex labeled with an acceptor and a long excited state lifetime luminescent donor, in which the changes in the donor lifetime are measured, has been described by Getz et al ((1998) Biophys J, 74:2451).

A single probe labeled with both donor and acceptor, where the energy transfer signal of the acceptor is measured in a TR-FRET assay, and where the signals from the reacted and non-reacted forms of the probe are separated using luminescence lifetime have not been described in the art.

### SUMMARY OF THE INVENTION

The present invention relates to a homogeneous bioaffinity assay based on the use a labelled probe comprising, a TR-FRET label pair comprising a long excited state lifetime luminescent energy donor and a short excited state lifetime luminescent energy acceptor, a reactive region, capable of recognizing a bioaffinity component to be determined. Said labelled probe, having different energy transfer efficiency between the donor and the acceptor when reacted to said bioaffinity component as compared to non-reacted probed, is contacted with a sample expected to comprise said bioaffinity component, and allowed to undergo a recognition reaction with said bioaffinity component. The energy transfer based signal from the acceptor of said reacted sample is measured and compared to the energy transfer based signal from the acceptor of a non-reacted probe in a reference assay; and the presence or absence of the bioaffinity component in the sample is determined based on a difference in the acceptor signals.

In one embodiment of the present invention the signal measurement is based on measurement of the lifetimes of obtained fluorescence signals.

In another embodiment of the present invention the signal measurement is performed as a time-gated intensity measurement, preferably in a time window, which is opened after a delay of at least 1 μs calculated from the donor excitation.

In one embodiment of the present invention, the measurement is performed in a time window which is opened after a short delay, preferably 1-25 μs, more preferably 1-10 μs, calculated from the excitation pulse.

In an other embodiment of the present invention, the measurement is performed in a time window which is opened after the short lifetime energy transfer based signal from the reacted probe has essentially decayed, preferably more than 25 μs, more preferably more than 50 μs.

According to another aspect, the invention concerns a labeled probe for use in bioaffinity assays, said probe comprising

a time-resolved fluorescence resonance energy transfer (TR-FRET) label pair comprising an energy donor and an energy acceptor,

a reactive region, capable of reacting with the bioaffinity component to be determined,

said probe having further the capability of changing the energy transfer efficiency between the donor and the acceptor upon reacting with the bioaffinity component.

According to the invention, the donor is preferably a long excited state lifetime luminescent label with excited state lifetime of at least 1 microsecond and the acceptor is a short excited lifetime luminescent label, and the probe is a nucleic acid, a polypeptide comprising 2 to 70 amino acids, or an antibody or antibody fragment.

### BRIEF DESCRIPTION OF THE DRAWINGS

FIGS. 1A and 1B show two examples of the time-gated measurement of the probe signal.

FIGS. 2A and 2B show the principle of dual analyte detection using the probe of the invention.

FIG. 3 shows the measured acceptor decay curves for hybridized and non-hybridized forms of the DNA probe in example 1.

FIGS. 4A and 4B show a simplified illustration on how the hybridization of the DNA probe with the target DNA hinders D-A interaction and affects the D-A distance.

FIG. 5 is a graphical presentation of the result of the test described in example 2. The figure shows the use of a DNA probe in time-gated intensity based detection of nucleic acid target.

FIG. 6 is a graphical presentation of the result of the test described in example 3. The figure shows the use of a DNA probe and the luminescence lifetime in the detection of nucleic acid target.

FIGS. 7A and 7B show the results of a hybridization assay described in example 4. in which the results of single-analyte and dual-analyte assays of two different DNA probes were compared.

FIGS. 8A and 8B show the principle of the helicase assay described in example 6. The assay utilizes a single-stranded DNA probe.

FIGS. 9A and 9B show the principle of the helicase assay described in example 7. The assay utilizes a double-stranded DNA probe.

### DETAILED DESCRIPTION OF THE INVENTION

The present invention provides a probe comprising an energy donor and an energy acceptor label and a reactive region capable of reacting with a bioaffinity component to be analyzed. The probe allows a simplified homogeneous and closed-tube detection of the bioaffinity component present in a sample or a test component. The present invention is based on the finding that upon reaction between the probe and the bioaffinity component the lifetime of the energy transfer signal changes. The energy transfer based acceptor signal of the reacted probe can be directly separated from the energy transfer based acceptor signal of the non-reacted probe without any need for additional assay steps or manipulation of the probe.

### Definitions and Preferred Embodiments

Bioaffinity Assay

The term “bioaffinity assay” shall mean a qualitative or quantitative method for determination of an analyte, i.e., a bioaffinity component in a sample, which can be a sample comprising a test compound, or a biological sample such as tissue sample, body fluid or other sample derived from a natural source such as the mammal body. Suitable bioaffinity assays include any recognition reactions based on binding, unbinding or any other modification of the labeled probe by the bioaffinity component to be determined.

Bioaffinity Component

“Bioaffinity component” shall mean an analyte present in a biological sample or a test component. The component can be, for example, a nucleic acid, such as DNA or RNA, a protein, such as an enzyme or antibody, a hapten, a carbohydrate or lipid or combinations thereof.

Probe

The terms “probe, decay resolvable probe, decay-resolved probe” shall be understood as a substance able to react with the bioaffinity component to be determined. As typical, non-limiting examples can be mentioned a nucleic acid or a modified nucleic acid (such as peptide nucleic acid, locked nucleic acid) probe. The term includes single- or double-stranded DNA, as well as single- or double-stranded RNA. The nucleic acid probe may include modified purine or pyrimidine bases. The nucleic acid probe comprises a sequence of preferably at least 6 nucleotides. Another preferred form of a probe is a polypeptide probe. The polypeptide probe may include modified amino acid residues. The polypeptide probe is preferably comprised of 2-70 amino acid residues. For easier production of the labeled probe, more preferably, the polypeptide probe contains 2-50 or only 2-40 amino acids. Even more preferably, the polypeptide probe may contain only 2-30 or 2-15 amino acids.

Reaction

The terms “reaction, reactive” refer most typically to a recognition reaction between the probe and the bioaffinity component to be determined. Examples of such reactions are nucleic acid hybridizations where Watson-Crick bonds are formed between single-stranded nucleic acid targets and probes; binding of proteins, such as antibodies or antibody fragments, to antigens, such as nucleic acid or polypeptide probes; binding of proteins, such as transcription factors, to nucleic acid probes. Moreover, the term “reaction” shall also be understood to include the opposite, namely cleavages of bonds, As a typical example, can be mentioned enzymatic cleavage of Watson-Crick bonds in double-stranded nucleic acid. Thus, in case the probe is a labelled double-stranded nucleic acid where the labels are located in the same strand, this probe can be used to determine the presence of an enzyme (e.g., helicase) in a sample. Moreover, the “reaction” shall also refer to a modification of the probe. Example of such a reaction is a polypeptide probe that can serve as a target for enzymatic modification. The “reactive region” of the probe refers to the specific part of the probe taking part in the reaction. Typically, the reactive region can be located between the donor and the acceptor but any other conformation is also possible. Thus, the reactive region can for example be located adjacent to the donor-acceptor pair.

Secondary Structure, Change of Secondary Structure

The term “secondary structure” refers to a structure of the probe that the probe obtains in different reaction conditions (the reacted or non-reacted forms of the probe). In addition to the reaction conditions, the secondary structure is also determined by the primary structure of the probe. The primary structure is determined by the nucleotide sequence of the nucleic acid probe and by the amino acid sequence of the polypeptide probe. A reaction of the probe with a bioaffinity component will most likely cause a change of said secondary structure of the probe. For example, the free non-reacted probe can have a “non-straight” such as curled/twisted/wounded shape, for example due to the interactions between the labels (illustrated in example 1), so that the labels are rather close to each other, and the reaction causes a straightening of the probe and thus an increase in the distance between the donor and the acceptor. As an example can be mentioned the binding reaction of a single-stranded nucleic acid with a complementary single-stranded nucleic acid (probe). This kind of reaction leads typically to straightening of the probe. As an example of a reaction leading to “non-straightening”, e.g. curling/twisting/winding of a probe, straight as non-reacted, when reacting with a bioaffinity component can be mentioned the unwinding of a double-stranded nucleic acid probe by helicase enzyme (illustrated in example 7). The change of secondary structure includes also cleavage, for example cleaving by an enzyme, of the probe. Moreover, change of secondary structure includes also modification, for example an enzymatic modification of an amino acid residue in the primary structure of the probe.

Label Interaction

Label interaction refers to an interaction that naturally occurs between the donor and the acceptor in the assay conditions and can affect to the D-A distance. As non-limiting examples can be mentioned stacking and charge interactions between the labels, which in the case of flexible probe structure can affect to the DA distance (example 1). In one embodiment of the present invention the specific reaction between the probe and the bioaffinity component can alter the effect of the label interaction.

FRET

FRET relates typically to Förster type of non-radiative dipole-dipole energy transfer between a fluorescent donor molecule and an acceptor molecule, which can be fluorescent or non-fluorescent. For certain label pair the critical energy transfer distance R₀ can be calculated using equation,

$${R}_{0} = {( 8.79 \cdot {10}^{- 5} \cdot J \cdot {q}_{D} \cdot {η}^{- 4} \cdot {κ}^{2} )}^{\frac{1}{6}} \qquad [ Å ]$$

where J is the spectral overlap integral between donor emission and acceptor absorption, q_{D} is the donor quantum yield, η is the refractive index of the medium and κ is factor related to the orientation between the donor and the acceptor.

Förster type energy transfer is highly dependent on the distance between the donor and the acceptor. The energy transfer efficiency E of a certain label pair is defined by the equation,
E=1/(1+r⁶/R₀⁶)
where r means the actual distance between the donor and the acceptor.

The R₀ defines the D-A distance, at which the energy transfer efficiency between the issued D-A label pair is 50%. The R₀-value of a D-A label pair can be used to roughly approximate the maximum usable D-A distance in the assay. For example D-A distance 1.44225*R₀ produces E=0.1 (only 10% energy transfer efficiency). The parameters in the R₀-equation represent the basic requirements for Förster type energy transfer and any change in these parameters will result a change in energy transfer of the issued D-A pair, even though the actual D-A distance remains unchanged. Typically, a large spectral overlap integral is considered as a rule of thumb when selecting efficient D-A pairs. The calculation of spectral overlap integral is described in the literature and is well-known to the person skilled in the art.

In this text, FRET shall also be considered to cover other energy transfer mechanisms, which are not necessarily fulfilling the requirements of Förster type energy transfer, but which are however capable to produce energy transfer based acceptor emission and the efficiency of the energy transfer can be affected by certain parameters.

TR-FRET

In TR-FRET the change in energy transfer is measured based on the lifetimes of obtained fluorescence signals or using time-gated intensity measurement, in which the fluorescence intensity of a sample is measured after a certain delay, calculated from the donor excitation pulse. The mean lifetime of the sample can also be measured comparing at least two time-gated intensity windows measured from the same sample. Time-gated intensity measurement (or lifetime based analysis) can be used to suppress the background in an assay, in which the lifetime of the background signal is shorter than the lifetime of actual energy transfer signal. Normally this involves the use of long lifetime donor (or/and acceptor) in the FRET pair. Special advantage is achieved when the lifetime of the free donor is remarkably longer than the lifetime of the free acceptor (τ_{D}>>τ_{A}). In this case the lifetime of the energy transfer based acceptor signal is determined as a function of the donor lifetime and energy transfer efficiency (see definition “energy transfer efficiency” below) and energy transfer based acceptor signal occurs in a very similar time scale with the donor. Time-gated intensity measurement (or lifetime analysis) can be used to eliminate the signal of directly excited acceptors, which are exited by the donor excitation pulse.

Luminescence Lifetime

In this text, terms luminescence lifetime, lifetime and decay time are synonyms for each other.

Energy Transfer Signal

In this text, the energy transfer based acceptor fluorescence is referred to as energy transfer signal.

Energy Transfer Efficiency

The energy transfer efficiency E and its relation to the lifetime of the energy transfer based acceptor signal in Förster type energy transfer is defined by the equation,
E=1/(1+r⁶/R₀⁶)=1−τ_{AD}/τ_{D},
where τ_{D} is the lifetime of the free donor and τ_{AD} is the lifetime of energy transfer based acceptor emission. The latter part of the equation is valid only when τ_{D}>>τ_{A}. The reaction of the probe with the bioaffinity component may cause some change in the distance between the donor and the acceptor. Thus the change of distance means also a change of E between the donor and the acceptor. A lengthening of the distance, r, results in a decreased E and vice versa. In general, any reaction, which affects the D-A distance, can be assumed to have certain effect on the energy transfer efficiency regardless of the exact mechanism of the issued energy transfer.

The distance between the labels is not the sole parameter that could be affected by the reaction. A change in any of the parameters defining the critical energy transfer distance R₀ (spectral overlap integral, donor quantum yield, refractive index of the medium, orientation factor) or generally change in fluorescent properties of the donor or acceptor or the environment around them will cause a change in the energy transfer efficiency.

Donors

The donor shall in this invention be a long excited lifetime luminescent label, which together with the short excited lifetime acceptor (τ_{D}>>τ_{A}) enables the energy transfer efficiency dependence of the lifetime of the induced acceptor signal. This further enables the time-gated intensity measurement of the acceptor signal and the luminescence lifetime based analysis of the induced acceptor signal. This means that the excited lifetime of the donor shall be at least 1 microsecond. Preferable are donors with 10 microseconds or more; particularly preferable are donors with lifetime of 100 microseconds or more.

As examples of preferable donors can be mentioned lanthanide chelates, up-converting phosphors (also called upconverting chelates), porphyrin complexes and metal chelate complexes based on the metal ions of groups VII and VIII of the transition elements. Suitable ligand structures for lanthanide chelates according to this invention are described for example in WO98/15830 and U.S. Pat. No. 5,998,146 and references cited therein. Upconverting phosphors have been described for example in U.S. Pat. No. 5,891,656 and their use in traditional homogeneous energy-transfer assays have been described for example in WO 2004086049.

Acceptors

The acceptor is preferably a highly luminescent molecule with quantum yield as near unity (1) as possible and with high molar absorption coefficient, preferably over 100 000. Preferably the acceptor has spectral overlap with the donor so that absorption spectrum of the acceptor overlaps with the emission spectrum of the donor. A preferred feature of the acceptor is its sharp emission at a wavelength where donor has minimum or does not have any emission. The lifetime of the acceptor shall be such that the emission of the directly excited acceptors should be totally decayed within 1 microsecond after the excitation pulse. Examples of suitable acceptors are disclosed in WO 98/15830 and the references cited therein but also many other acceptors are known in the art.

Time Window

A person skilled in the art realizes that when τ_{D}>>τ_{A} the lifetime of the energy transfer signal is defined as a function of τ_{D} and the energy transfer efficiency from the donor to the acceptor. This phenomenon is independent of the exact mechanism of the energy transfer and a change in the energy transfer efficiency can always be assumed to cause a change in the lifetime of energy transfer signal.

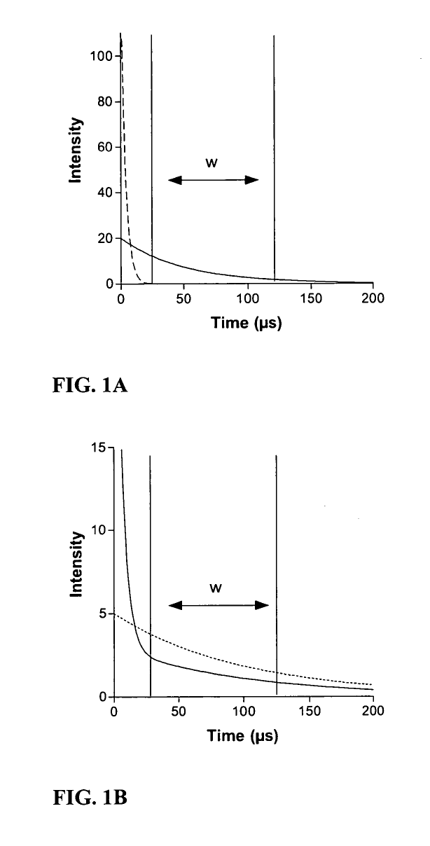

In Förster type FRET the lifetime of the energy transfer signal is defined according to equation,
E=1−τ_{AD}/τ_{D} (valid when τ_{D}>>τ_{A})
where E is energy-transfer efficiency, τ_{AD} is the lifetime or decay of the energy transfer signal and τ_{D} is the lifetime of the free donor). If the reaction, for example, results in a decrease of E, this will in turn lead to a prolonged τ_{AD} compared to the acceptor decay of the non-reacted probe. This matter of fact enables the discrimination between the reacted probe and the non-reacted probe using a time-gated intensity measurement or luminescence lifetime measurement. The principle of time-gated measurement is illustrated in FIG. 1A. The time-gated measurement window (TR window), w, is opened using a delay time, after which the energy transfer signal of non-reacted probe (dashed line) has decayed and the energy transfer signal of reacted probe (solid line) can be detected without background. Alternative option is to use very short delay time (1-10 μs) and a TR window, in which both signals can be detected simultaneously. Reaction of the probe will cause a decrease in the signal.

If the reaction of the probe causes an increase of E, this will lead to a two-exponential acceptor decay, in which reacted probes cause a population with shortened lifetime compared to the lifetime of the non-reacted probe population, FIG. 1B. In this case the TR window (w) can be opened using a delay time, after which the short lifetime signal of the reacted probe population has decayed and the decrease in the long lifetime signal of the sample (solid line) compared to the control sample signal (dashed line) is measured. Alternative option is to use very short delay time (1-10 μs) and a TR window, in which both signals can be detected simultaneously. Reaction of the probe will cause an increase in the signal.

Multianalyte Detection

The probe of the invention can also be used to carry out a simple multianalyte assay, in which multiple analytes can be detected simultaneously from the same assay medium. Multianalyte assay is possible when each analyte specific probe according to this present invention generate:

(1) Clearly spectrally resolvable emissions and thus different analyte signals can be separated using optical filtering.

(2) Emissions on the same wavelength but with different, resolvable, lifetimes. The analyte signals can be separated using luminescence lifetime fitting.

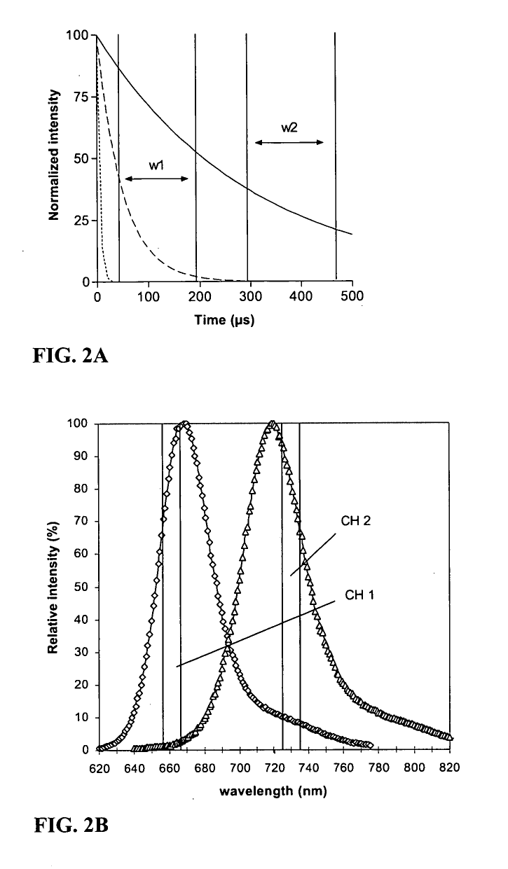

(3) Emissions partially on the same wavelength (crosstalk) but with different lifetimes. The analyte signals can be separated using luminescence lifetime fitting or using time-gated intensity measurement together with optical filtering. This particular case is shown in FIGS. 2A and 2B. In 2A the solid line corresponds to analyte 2 decay, the dashed line corresponds to analyte 1 decay and the dotted line is the decay of the non-reacted probes. In 2B open diamonds correspond to analyte 1 emission spectrum and open triangles correspond to analyte 2 emission spectrum. Analyte 1 has emission in the optical channel of analyte 2 (CH 2) but the crosstalk is eliminated using appropriate TR-window for analyte 2 (w2). The signal of analyte 2 exists in the TR window of analyte 1 (w1) but crosstalk is eliminated using appropriate optical channel for analyte 1 (CH 1).

Luminescence Lifetime Analysis

If time-gated intensity measurement is not used, the signals of the reacted and non-reacted probes can be resolved with luminescence lifetime analysis of the sample. Time-domain or frequency-domain measurements are typically used for the lifetime determination. Both measurement techniques, their applications and the related data analysis methods are well described in the literature and are well-known to the person skilled in the art. The lifetime analysis can be applied to a decay resolvable probe based assay, for example, using the following method: The decay characteristics of a probe, specific to the assay, are first predetermined measuring the decay times of the reacted and non-reacted probe (positive sample and reference sample, respectively). The non-reacted probe will produce single-exponential decay time τ_{non-reacted}. The positive sample typically decays two-exponentially with decay times τ_{non-reacted} and τ_{reacted} or single exponentially with decay time τ_{reacted} only. The actual assay samples are then measured and the obtained data is fitted to a suitable lifetime-equation using pre-determined decay times τ_{non-reacted} and τ_{reacted} as constants in the fitting. Most commonly decay data is fitted to equation,

$$I ( t ) = {\sum }_{i} {A}_{i} {ⅇ}^{- \frac{t}{{τ}_{i}}}$$

where l(t) is the intensity of the sample on a certain moment of time. The term Aᵢ corresponds to the size of the luminescent population emitting with decay time τᵢ. In the case of single analyte assay, the result is two-exponential fitting, which produces population parameters A_{reacted} and A_{non-reacted}. The assay result is resolved comparing the population parameters of the positive sample and reference sample. In the case of multianalyte assay, each probe is preferably designed to have different decay time, when reacted with the target. Each analyte will then produce its own exponential term to the luminescence data and the assay result is resolved using multi-exponential fitting.

Merely the presence of a certain decay time component in the sample signal is a qualitative measure for the existence of the bioaffinity component in the sample.

These examples are given to illustrate the lifetime based analysis of the assay, but were not intended to limit the scope of the invention. Also other lifetime analysis methods are applicable.

### ADVANTAGES OF THE INVENTION

The decay resolvable probe of the invention enables a simple assay, in which the time-resolved homogeneous detection of the analyte can be carried out using only one additional reagent (the probe) per analyte. A homogeneous assay for a certain target of interest is therefore possible to carry out using only one assay step. The probe according to the present invention comprises both donor and acceptor labels and no additional steps, such as cleavage or other manipulation of the non-reacted or reacted probes, are required to resolve the assay results.

The probe design of the invention enables also an easy way to carry out homogeneous multianalyte assays based on the emission of energy transfer signal. The use of only one bioaffinity compound per analyte is an advantage when concerning the selectivity and sensitivity of an assay.

### EXAMPLES

The following examples are given to further illustrate preferred embodiments of the present invention, but are not intended to limit the scope of the invention. It will be obvious to a person skilled in the art, as the technology advances, the inventive concept can be implemented in various ways. The invention and its embodiments are not limited to the examples described above but may vary within the scope of the claims.

### Example 1

Decay Resolvable DNA Probe

This example demonstrates a single-stranded DNA probe according to the present invention, in which the energy transfer efficiency is different between the reacted and non-reacted form of the probe. In this particular case, the specific reaction with the target decreases the energy transfer efficiency.

DQB1*0302 target-specific probe (5′ TTT CCG CCT GCC GC 3′) was labeled to its 5′ end with a stable fluorescent W8194 Eu chelate (PerkinElmer Life and Analytical Sciences, Wallac) and to its 3′ end with an organic fluorophore Alexa Fluor 647 (Molecular Probes). Hybridization of 10 nM DQB1*0302 probe and 2 nM DQB1*0302 target (5′ CTC GGC GGC AGG CGG CCC CAC GTC GCT GTC GM GCG CAC GAT CTC TTC T 3′) was performed in a total volume 200 μl containing 75 mM Tris-Cl (pH 8.5), 50 mM KCl, 20 mM (NH₄)₂SO₄, 3 mM MgCl₂, 0.1% Triton X-100. Hybridization reaction was first incubated at 40° C. for 30 min and then at 22° C. for 30 min, after which the sample was pipetted to a microtiter plate, 25 μl/well (1508-0010, PerkinElmer Life and Analytical Sciences). Time-resolved measurements were made on a laboratory build TR-fluorometer utilizing nitrogen laser (model 79111, Oriel, <10 ns pulse, 45 Hz), photomultiplier tube (Hamamatsu) and Turbo MCS multichannel scaler (EG&G) with 0.1 μs time resolution. Energy transfer based emission was collected through 665 nm emission filter (Omega Optical, band width 7 nm).

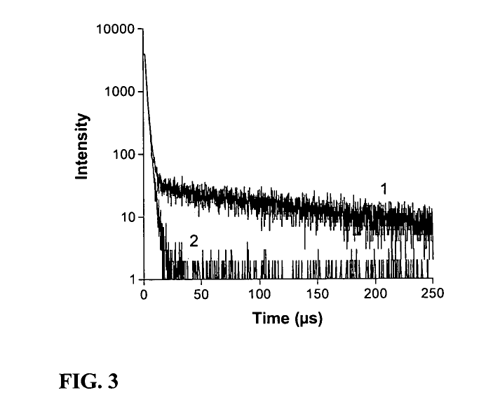

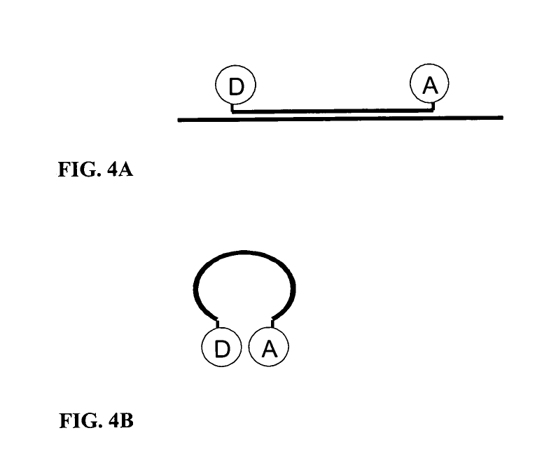

Fluorescence decays of positive sample (probe+target, curve 1) and negative control (probe only, curve 2) are shown in FIG. 3. Negative control has mono-exponential decay with lifetime of 1.7 μs. Positive sample has two-exponential decay with lifetime components 1.7 μs and 165 μs. The long lifetime component is caused by the hybridized probe population, in which the D-A distance is forced to be long due to the rigid double helix structure between the labels, FIG. 4A. In non-reacted probe, the strong interaction (natural), which occurs between the donor Eu and Alexa Fluor 647, is capable to bring the labels in close proximity due to the flexibility of single-stranded DNA chain. FIG. 4B. The energy transfer signal of reacted probe can be easily resolved from the energy transfer signal of non-reacted probe using time-gated intensity measurement (FIG. 1A) with appropriate delay time (e.g. 50 μs for this probe).

### Example 2

Nucleic Acid Analysis Using a Decay Resolvable DNA Hybridization Probe and Time-Gated Intensity Measurement

This example shows the proof of principle of a DNA assay using the decay resolvable DNA hybridization probe. The probe is allowed to hybridize with a complementary target, after which the amount of hybridized probe corresponding to the amount of target can be measured in a time-resolved manner. It is shown that the detection technology can be used for the detection of target nucleic acid.

Hybridization reaction was performed in a total volume of 50 μl containing 75 mM Tris-Cl (pH 8.5), 50 mM KCl, 20 mM (NH₄)₂SO₄, 3 mM MgCl₂, 0.1% Triton X-100, different amounts of DQB1*0302 target (5′ CTC GGC GGC AGG CGG CCC CAC GTC GCT GTC GAA GCG CAC GAT CTC TTC T 3′), and 10 nM DQB1*0302 specific probe (5′ TTT CCG CCT GCC GC 3′) labeled at its 5′ end with a stable fluorescent W8194 Eu chelate (PerkinElmer Life and Analytical Sciences, Wallac) and at its 3′ end with an organic fluorophore Alexa Fluor 647 (Molecular Probes). Hybridization reactions were first incubated at 40° C. for 30 min and then at 22° C. for 30 min, after which the fluorescence was measured with Victor™ V 1420 Multilabel Counter (PerkinElmer Life and Analytical Sciences). Measurement parameters are shown below.

| Parameter | VictorV |
|---|---|
| Excitation (nm) | 340 |
| Emission (nm) | 665 |
| Delay time (μs) | 50 |
| Window time (μs) | 50 |
| Cycle (μs) | 1000 |

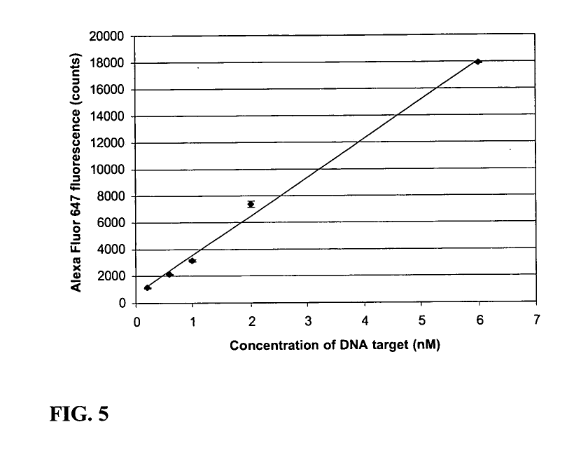

The average acceptor fluorescence and standard deviation of three replicates of each sample are shown in FIG. 5. The assay has linear response for different amounts of nucleic acid target and proves that the detection principle can be used for the detection of nucleic acids. The assay yielded signal-to-background ratio of 3.3 for 0.2 nM of target DNA. In this example, the specific reaction between the probe and the target decreases the energy transfer efficiency. Moreover, the measurement can be performed with present instrumentation (i.e. Victor™ Multilabel Counter).

### Example 3

Nucleic Acid Analysis Using Decay Resolvable DNA Hybridization Probe and Luminescence Lifetime Measurement

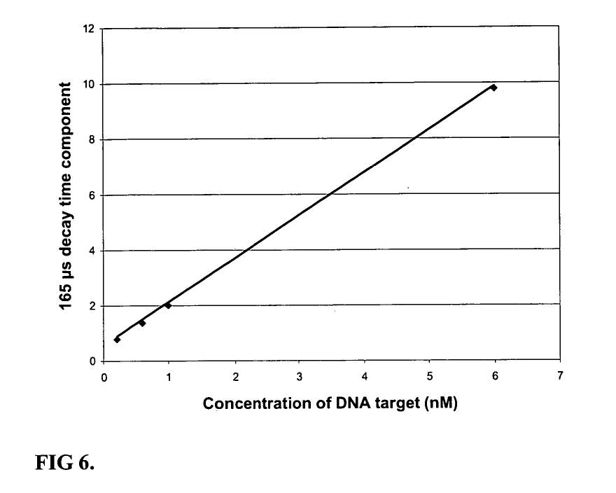

The assay according to the example 2 was measured using the TR-fluorometer described in example 1. The measured fluorescence decay curves of the samples were fitted according to equation:

$$I ( t ) = {A}_{1} {ⅇ}^{- \frac{t}{{τ}_{1}}} + {A}_{2} {ⅇ}^{- \frac{t}{{τ}_{2}}}$$

where l(t) is the is the intensity of the sample on a certain moment of time, and A₁ and A₂ are factors proportional to the sizes of the fluorescent populations having decay times τ₁ and τ₂, respectively. Data was fitted using decay time parameters 1.7 μs and 165 μs determined in example 1. The average value of the 165 μs decay time component (A₂) of each sample is shown in FIG. 6. The lifetime measurement based analysis of the data has linear response for different amounts of nucleic acid target and yielded signal-to-background ratio of 5.4 for 0.2 nM of target DNA. The result proves that the lifetime measurement based analysis of the data produces similar results with the time-gated intensity detection and is an alternative method to measure the assay.

### Example 4

Dual-Analyte Nucleic Acid Analysis Using Decay Resolvable Hybridization Probes

This example shows the proof of principle of a dual-analyte DNA assay using decay resolvable hybridization probes. It is shown that two decay resolvable probes labeled with the same donor but with different acceptors can be used for the dual-analyte detection.

Hybridization reaction was carried out as described in the example 2 including 10 nM of the first decay resolvable hybridization probe (DQB1*0302 probe 5′ TT CCG CCT GCC GC 3′) labeled at its 5′ end with a stable fluorescent W8194 Eu chelate (PerkinElmer Life and Analytical Sciences, Wallac) and at its 3′ end with an organic fluorophore Alexa Fluor 647 (Molecular Probes) and 10 nM of the second decay-resolved hybridization probe (DQA1*control probe 5′ TCT CCA TCA AAT TCA T 3′) labeled at its 5′ end with a stable fluorescent W8194 Eu chelate (PerkinElmer Life and Analytical Sciences, Wallac) and at its 3′ end with an organic fluorophore Alexa Fluor 700 (Molecular Probes). The first and second probes are designed to hybridize with their own specific complementary targets (DQB1*0302 target 5° CTC GGC GGC AGG CGG CCC CAC GTC GCT GTC GAA GCG CAC GAT CTC TTC T 3′ and DQA1*control target 5′ CCATGAATTTGATGGAGATGTCTGGAAGTTGCC 3′), and after hybridization the fluorescence is measured with Victor™ V 1420 Multilabel Counter (PerkinElmer Life and Analytical Sciences). The fluorescence signals proportional to the hybridized first and second probes were measured using the following measurement parameters:

| | Alexa Fluor 647 | Alexa Fluor 700 |
|---|---|---|
| Parameter | measurement | measurement |
| Excitation (nm) | 340 | 340 |
| Emission (nm) | 665 | 730 |
| Delay time (μs) | 50 | 400 |
| Window time (μs) | 50 | 400 |
| Cycle (μs) | 1000 | 1000 |

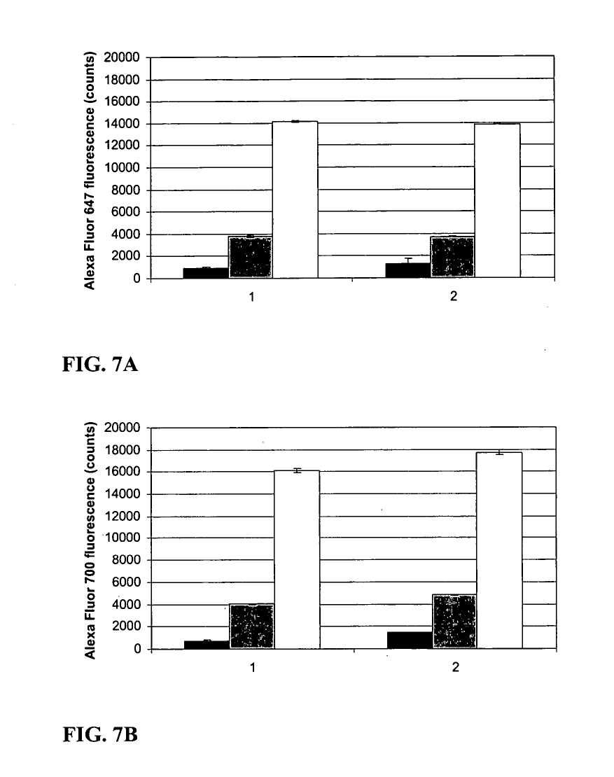

FIGS. 7A and 7B show the average and standard deviation of acceptor fluorescence counts of three replicas from single-analyte (1) and dual-analyte (2) hybridization assays. Black, grey, and white bars show the fluorescence signals obtained from reactions containing no target DNA (blank), 1 nM target DNA, and 5 nM target DNA, respectively. FIG. 7A presents the results obtained with Alexa Fluor 647 labeled probe in single-analyte assay (1) containing only the DQB1*0302 probe and DQB1*0302 DNA target, and in dual-analyte assay (2) containing the DQB1*0302 and DQA1*control probes, and DQB10302 and DQA1*control DNA targets. FIG. 7B presents the results obtained with Alexa Fluor 700-labeled probe in single-analyte assay (1) containing only the DQA1*control probe and DQA1*control DNA target, and in dual-analyte assay (2) containing the DQB1*0302 and DQA1*control probes, and DQB1*0302 and DQA1*control DNA targets. In this case, the specific reactions between the probe and the target decrease the energy transfer efficiency. Both probes yielded similar results both in the single-analyte and dual-analyte assays and thus can be concluded that the decay resolvable probes can also be used for dual-analyte detection.

### Example 5

A Decay Resolvable Peptide Probe

This example is to illustrate that the decay resolvable probe can be a labeled polypeptide probe, more specifically a labeled peptide probe. Here, the peptide probe serves as an antigen containing an epitope for antibody binding. Antibody binding to the probe introduces a conformational change in the labeled probe thus allowing the separation of reacted and nonreacted probe.

A peptide probe containing a phosphotyrosine residue (pY) served as an antigen to anti-phosphotyrosine antibody. The peptide, Ac-CGGGGpYGGGG-NH2, is labeled with iodoacetamino-activated fluorescent europium chelate and with Alexa Fluor 647 carboxylic acid succinimidyl ester into the cysteine and and free amino group of the peptide, respectively. Fifty-microliter reactions in 50 mM Tris, 150 mM NaCl, pH 7.75 with 10 nM labeled peptide with or without 1 nM anti-phosphotyrosine antibody are incubated at room temperature for 30 minutes with slow shaking. After incubation, the energy transfer signal is measured.

Binding of anti-phosphotyrosine antibody with the phosphotyrosine in the peptide probe changes the conformation of the probe and thus the reacted and non-reacted forms of the probe can be separated based on the different acceptor fluorescence emission lifetimes of these two forms. In this case, the binding reaction decreases the energy transfer efficiency. Peptide probes can be exploited for example in enzyme activity assays (e.g., kinase assay) or in competitive homogeneous immunoassays (e.g., peptide hormone assays).

### Example 6

Helicase Assay with a Decay Resolvable Single-Stranded DNA Capture Probe

This example utilizes a doublestranded DNA substrate containing a structure that is recognized by helicase enzyme, and a single-stranded, labeled decay resolvable capture probe that is labeled at the 5′ end with a donor label and at the 3′ end with an acceptor label. The double-stranded helicase substrate contains two complementary oligonucleotide strands, the sense strand consisting of 44 bases and the antisense strand consisting of 26 bases. The labeled capture strand of 16 bases is complementary to the antisense strand of the substrate.

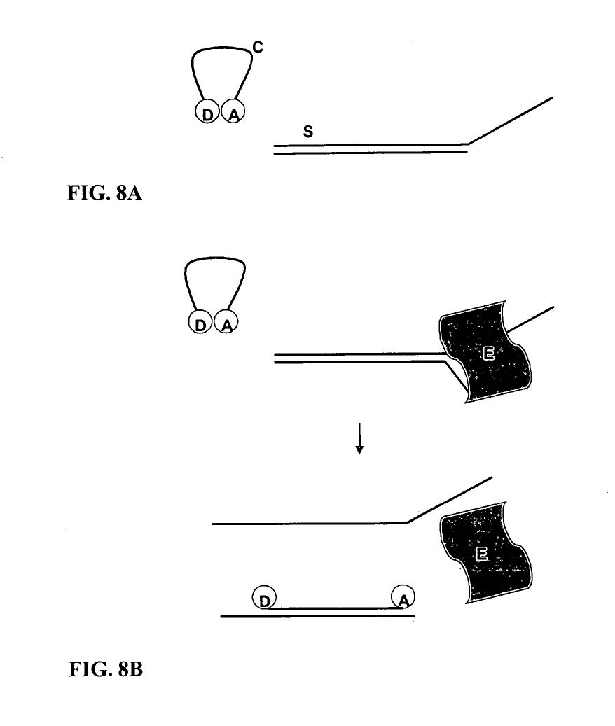

Principle of the assay is shown in FIG. 8. In the absence of enzyme activity (FIG. 8A), the substrate (S) remains in the double-stranded form and the single-stranded, labeled, decay-resolved capture probe (C) is free in the solution and having conformation 1. In conformation 1, the donor (D) and acceptor (A) labels are brought to close proximity resulting in efficient energy transfer and short luminescence lifetime. In the presence of enzyme activity (FIG. 8B), the helicase (E) unwinds the double-stranded substrate (S) and yields single-stranded intermediates. Upon unwinding of the doublstranded DNA, the labeled capture probe obtains conformation 2 by hybridizing with the single-stranded, 26-mer antisense strand. In conformation 2, the D-A distance is longer and thus the luminescence lifetime of the energy transfer signal is longer than in the case of non-reacted capture probe (conformation 1).

### Example 7

Helicase Assay With a Decay Resolvable Double-Stranded DNA Probe as an Enzyme Substrate

This example utilizes a labeled, double-stranded DNA substrate containing a structure that is recognized by helicase enzyme, and a single-stranded capture strand. The double-stranded helicase substrate contains two complementary oligonucleotide strands, the labeled sense strand consisting of 44 bases and the antisense strand consisting of 26 bases. The capture strand contains 16 bases and is complementary to the antisense strand of the substrate. The 44-mer sense strand of the substrate is labeled at its 5′ end with a fluorescent europium chelate (donor) and 10 bases downstream from the 5′ end with an acceptor label. The acceptor label is introduced to an amino linker of a modified base that retains its capability of forming hydrogen bonds with a complementary base.

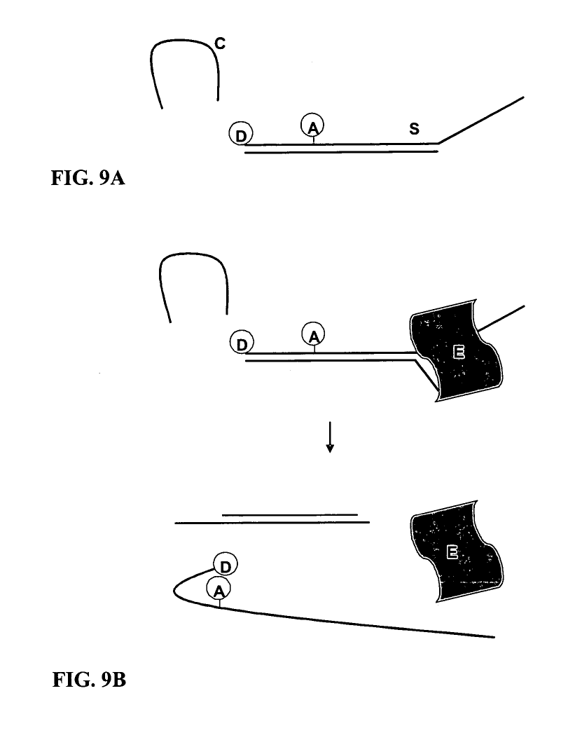

Principle of the assay is shown in FIG. 9. In the absence of enzyme activity (FIG. 9A), the substrate (S) remains in conformation 1, that is, in the doublestranded form keeping the donor (D) and the acceptor (A) labels 10 bases apart from each other. In the presence of helicase activity (FIG. 9B), the enzyme (E) unwinds the double-stranded substrate and yields two single strands. The capture strand (C) hybridizes with the single-stranded antisense strand of the substrate, and the labeled, single-stranded 44-mer sense

1. A homogeneous bioaffinity assay comprising the steps of
a) providing a labelled probe comprising
(i) a TR-FRET label pair comprising a long excited state lifetime luminescent energy donor and a short excited state lifetime luminescent energy acceptor; and
(ii) a reactive region, capable of recognizing a bioaffinity component to be determined;
b) contacting said labelled probe with a sample, expected to comprise said bioaffinity component;
c) allowing said probe and said bioaffinity component to undergo a recognition reaction; said probe having different energy transfer efficiency between the donor and the acceptor when reacted to said bioaffinity component as compared to non-reacted probe;
d) measuring an energy transfer based signal from the acceptor of said reacted sample;
e) measuring an energy transfer based signal from the acceptor of a non-reacted probe in a reference assay;
f) comparing the signals obtained in steps d) and e); and
g) determining the presence or absence of the bioaffinity component in the sample based on a difference in acceptor signals obtained in step (f).

2. The assay according to claim 1, wherein the measurement in steps d) and e) is based on measurement of the lifetimes of obtained fluorescence signals.

3. The assay according to claim 1, wherein the measurement in steps d) and e) is performed as a time-gated intensity measurement.

4. The assay according to claim 3, wherein the time-gated intensity measurement of said energy transfer based signal from the acceptor is performed in a time window which is opened after a delay of at least 1 microsecond calculated from the donor excitation.

5. The assay according to claim 4, wherein the recognition reaction between the probe and the bioaffinity component in step c) results in a decrease of the energy transfer efficiency between the donor and the acceptor;
wherein said time-gated intensity measurement is performed in a time window which is opened after the energy transfer based signal, obtained from the acceptor in a reference assay carried out on the non-reacted probe, essentially has decayed, and
wherein step g) is performed using an increased acceptor signal, compared to the reference signal in the same window, as indication of the presence of the bioaffinity component in the sample.

6. The assay according to claim 4, wherein the recognition reaction between the probe and the bioaffinity component in step c) results in a decrease of the energy transfer efficiency between the donor and the acceptor;
wherein said time-gated intensity measurement is performed in a time window which is opened after a short delay before the acceptor signal measured from the reference assay carried out on the non-reacted probe has decayed; and
wherein step g) is performed using a decreased acceptor signal, compared to the reference signal in the same window, as indication of the presence of the bioaffinity component in the sample.

7. The assay according to claim 4, wherein the recognition reaction between the probe and the bioaffinity component in step c) results in increase of the energy transfer efficiency between the donor and the acceptor;
wherein said time-gated intensity measurement is performed in a time window which is opened after the short lifetime energy transfer based signal resulting form the reacted probes, essentially has decayed; and
wherein step g) is performed using a decreased acceptor signal, compared to the reference assay signal in the same window, as indication of the presence of the bioaffinity component in the sample.

8. The assay according to claim 4, wherein the recognition reaction between the probe and the bioaffinity component in step c) results in increase of the energy transfer efficiency between the donor and the acceptor;
wherein said time-gated intensity measurement is performed in a time window which is opened after a short delay before the short lifetime component of the acceptor signal measured from the reacted probe has decayed; and
wherein step g) is performed using an increased acceptor signal, compared to the reference assay signal in the same window, as indication of the presence of the bioaffinity component in the sample.

9. The assay according to claim 2, wherein decay data of said time-resolved energy transfer based signal from the acceptor is measured; said decay data is fitted to a luminescence lifetime equation to resolve the decay time parameters of said sample signal; said decay time parameters are compared to decay time parameters obtained from a reference assay carried out on the non-reacted probe; and the presence or absence of said bioaffinity component in said sample is determined based on differences in said decay parameters.

10. The assay according to claim 1, wherein said bioaffinity assay format is selected from the group consisting of a nucleic acid hybridization, an antibody-antigen assay, and an enzyme activity assay.

11. A labelled probe for use in a bioaffinity assay, said probe comprising
a) a time-resolved fluorescence resonance energy transfer (TR-FRET) label pair comprising an energy donor and an energy acceptor; and
b) a reactive region, capable of recognizing a bioaffinity component to be determined, said probe having different energy transfer efficiency between the donor and the acceptor when reacted to said bioaffinity component as compared to non-reacted probe, wherein
said donor is a long excited state lifetime luminescent label and said acceptor is a short excited lifetime luminescent label; said probe being selected from the group consisting of a nucleic acid, a polypeptide comprising 2 to 70 amino acids, an antibody and an antibody fragment; and
said donor is a luminescent label having an excited state lifetime of at least 1 microsecond.

12. The probe according to claim 11, wherein said difference in energy transfer efficiency between the donor and the acceptor when said probe is reacted to said bioaffinity component as compared to non-reacted probe is caused by a change of the secondary structure of said probe.

13. The probe according to claim 12, wherein said energy transfer efficiency is decreased when said probe is reacted to said bioaffinity component.

14. The probe according to claim 12, wherein said energy transfer efficiency is increased when said probe is reacted to said bioaffinity component.

15. The probe according to claim 11, wherein said probe is a nucleic acid having a reactive region which is a nucleotide sequence complementary to a nucleotide sequence of a target region in said bioaffinity component.

16. The probe according to claim 11, wherein said probe is a nucleic acid, which is a substrate in said bioaffinity assay.

17. The probe according to claim 11, wherein said probe is a polypeptide, capable of reacting as a target for enzymatic modifications or as an antigen.

18. The probe according to claim 19, wherein said probe is an antibody or antibody fragment, capable of binding to an epitope of an antigen.
